(19) United States
(12) Patent Application Publication
(10) Pub. No.: US 2003/0013694 A1
(43) Pub. Date: Jan. 16, 2003

(54) Use and compositions of antiprogestins for treatment of prostate diseases

(76) Inventors: Jens Hoffmann, Muehlenbeck (DE); Ulrike Fuhrmann, Berlin (DE); Rosemarie Lichtner, Berlin (DE); Martin Schneider, Berlin (DE); Gerhard Siemeister, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 10/153,856

(22) Filed: May 24, 2002

Related U.S. Application Data
(60) Provisional application No. 60/293,194, filed on May 25, 2001.

(51) Int. Cl. A61K 31/56

(52) U.S. Cl. 514/179

(57) ABSTRACT

The present invention relates to methods and uses for preventing and treating prostate diseases, in particular prostate cancer and benign prostate hyperplasia, in male mammals by antiprogestins, in particular antiprogestin 11β-(4-acetylphenyl)-17β-hydroxy-17α-(1,1,2,2,2-pentafluoroethyl)-estra-4,9-dien-3-one or a pharmaceutically acceptable derivative or analogue thereof. The invention further relates to pharmaceutical compositions comprising said antiprogestin.

This application claims the benefit of the filing date of U.S. Provisional Application No. 60/293,194 filed May 25, 2001.

### FIELD OF THE INVENTION

The present invention relates to the use of antiprogestins for the prophylaxis and treatment of prostate diseases like prostate cancer and benign prostate hyperplasia. In particular, the invention relates to the use of the antiprogestin 11β-(4-acetylphenyl)-17β-hydroxy-17α-(1,1,2,2,2-pentafluoroethyl)-estra-4,9-dien-3-one or a pharmaceutically acceptable derivative or analogue thereof for the treatment of such diseases. The invention also includes pharmaceutical compositions effective for the treatment of prostate diseases.

### BACKGROUND OF THE INVENTION

The androgen receptor is involved in the development, growth and progression of prostate cancer. Accordingly, treatment of prostate cancer by the administration of antiandrogens was the standard therapeutic approach in the past. However, in many cases, prostate carcinoma cannot be treated by the sole administration of antiandrogens.

WO 91/00733 discloses a method for treating androgen-related diseases such as prostate cancer, the method comprising administering certain antiandrogens and/or sex steroid biosynthesis inhibitors as part of a combination therapy. The sex steroid biosynthesis inhibitors include both androgen and estrogen formation inhibitors. According to the teaching of WO 91/00733, antiestrogens can also be part of the combination therapy.

WO 94/27610 teaches the treatment of prostate carcinoma and benign prostate hyperplasia (BPH) by the administration of a combination of at least one compound having antiandrogen activity and at least one compound having competitive, progesterone antagonist activity. WO 94/27610 further discloses the use of a compound having competitive progesterone antagonist activity alone for the prophylaxis and treatment of BHP.

Although antiprogestins were originally created with regard to medicinal non-surgical termination of pregnancy, certain antiprogestins have recently gained importance in a panel of other therapeutic applications beside the BHP treatment mentioned above, e.g. the endocrine therapy of progesterone-receptor positive breast cancers (T. Maudelonde et al., in J. G. M. Klijn et al., Hormonal Manipulation of Cancer: Peptides, Growth Factors and New (Anti) Steroidal Agents, Raven Press, New York, 1987, pp. 55-59). In certain prostate cancer models (such as R3327H), the antiprogestin mifepristone (RU-486; EP-A-0 057 115) was reported to effect substantial inhibition of tumor growth when administered alone (B. Mobbs et al., J. of Steroid Biochem. Mol. Biol, 39, pp. 713-722, 1991). Recently published data indicates that mifepristone (RU-486) and tamoxifen are effective inhibitors of apoptosis in vitro and in vivo (El Etreby et al., Prostate 2000, February 1, 42(2), pp. 99-106; ibid., April 1, 43(1) pp. 31-42) and may represent non-androgen ablation, novel therapeutic approaches to overcome a potential intrinsic apoptosis resistance of androgen-independent prostate cancer cells.

Prostate cancer often progresses from an androgen-dependent to an androgen-independent tumor, making androgen ablation therapy ineffective. The mechanisms for the development of androgen-independent prostate cancer are still unclear. More than 80% of clinically androgen-independent prostate tumors show high levels of androgen receptor expression (J. Trapman, Path. Res. Pract. 192, pp. 752-760 (1996)). In prostate tumors that have relapsed from androgen-ablation therapies the androgen receptor is still expressed and, compared to the primary tumors, its level is often even enhanced. Mutated androgen receptors can be activated by other compounds such as adrenal steroids, estrogens, progestins, even antiandrogens and furthermore, glucocorticoids (X.-Y. Zhao, Nature Medicine, Vol. 6, No, 6, June 2000, pp. 703-706). Relapse of tumors under the selective pressure of common androgen-ablation therapies can be caused by acquired androgen hypersensitivity and androgen receptor activation by ligands other than (dihydro)testosterone.

Thus, there is a clear-felt clinical need for compounds that are effective inducers of apoptosis in recurrent prostate tumors. A new therapeutic approach that is not directed towards the androgen receptor is desired to provide effective treatment of prostate cancer and other related diseases such as benign prostate hyperplasia.

### OBJECT OF THE PRESENT INVENTION

It is an object of the present invention to prevent or reduce the disadvantages of the prior art by providing an improved treatment for prostate cancer, whether androgen-dependent, androgen-independent or partially androgen-dependent, as well as a superior treatment for benign prostate hyperplasia and other prostate diseases, In this context it is especially desired to provide a treatment of prostate diseases, which avoids serious and risk-laden surgery commonly involved with castration. It is further desirable to provide a treatment for androgen-independent prostate cancer subtypes for which no proper treatment has existed before.

It is a further object of the present invention to provide pharmaceutical compositions comprising highly effective agents for the treatment of prostate diseases such as prostate cancer and benign prostate hyperplasia.

Another object of the present invention is the provision of a treatment of prostate diseases with increased oral bioavailability in order to reduce side effects related to high dosage and to ensure a targeted treatment.

These objects are surprisingly achieved by the use of antiprogestins according to the present invention and by pharmaceutical compositions and dosage forms comprising such antiprogestins. Particularly preferred in this regard is the antiprogestin 11β-(4-acetylphenyl)-17β-hydroxy-17α-(1,1,2,2,2-pentafluoroethyl)-estra-4,9-dien-3-one or a pharmaceutically acceptable derivative or analogue thereof, optionally in combination with further therapeutically active agents.

### SUMMARY OF THE INVENTION

The present invention is based on the novel and unexpected observation that certain antiprogestins, in particular the antiprogestin 11β-(4-acetylphenyl)-17β-hydroxy-17α-(1,1,2,2,2-pentafluoroethyl)-estra-4,9-dien-3-one or a pharmaceutically acceptable derivative or analogue thereof exert an inhibiting effect on tumor development in a panel of prostate tumor models. The antiprogestins, in particular antiprogestin 11β-(4-acetylphenyl)-17β-hydroxy-17α-(1,1,2,2,2-pentafluoroethyl)-estra-4,9-dien-3-one or a pharmaceutically acceptable derivative or analogue thereof turned out to be superior to known treatments, e.g. antiandrogens and even to castration commonly applied to treat prostate diseases, such as prostate cancer. Accordingly, the preferred antiprogestin according to the invention, i.e., the antiprogestin 11β-(4-acetylphenyl)-17β-hydroxy-17α-(1,1,2,2,2-pentafluoroethyl)-estra-4,9-dien-3-one (hereinafter referred to as “antiprogestin (I)”) or a pharmaceutically acceptable derivative or analogue thereof is distinctly more potent in inhibiting prostate tumor growth than certain antiandrogens, e.g. casodex.

In a first aspect, the present invention provides the use of antiprogestin (I) or a pharmaceutically acceptable derivative or analogue thereof for the preparation of a medicament for the treatment of prostate diseases in male mammals, preferably male humans. Examples of such prostate diseases are prostate cancer and benign prostate hyperplasia, whereby prostate cancer is the most preferred indication.

In a second aspect, the present invention provides a method for treatment of prostate diseases in male mammals, preferably male humans, in need of such treatment. The method comprises administering a pharmaceutically effective amount of an antiprogestin, in particular antiprogestin (I) or a pharmaceutically acceptable derivative or analogue thereof, to a male mammal in need thereof. Examples of such prostate diseases are prostate cancer and benign prostate hyperplasia, whereby prostate cancer is the roost preferred indication.

In a third aspect, the present invention further provides pharmaceutical compositions comprising an antiprogestin, in particular antiprogestin (I) or a pharmaceutically acceptable derivative or analogue thereof, in an amount sufficient for the treatment of prostate diseases. Examples of such prostate diseases are prostate cancer and benign prostate hyperplasia, whereby prostate cancer is the most preferred indication.

In a fourth aspect, the present invention provides a use/method/pharmaceutical composition for a combination therapy for prostate diseases, such as prostate cancer or benign prostate hyperplasia. Aside from the antiprogestin (I) or a pharmaceutically acceptable derivative or analogue thereof, the combination therapy may include at least one further pharmacologically active agent, e.g. at least one antiandrogen, antiestrogen, estrogen or other agents listed in the detailed description of the invention. Furthermore, therapy combinations are also envisaged, such as combination of the antiprogestin therapy according to the invention with a chemotherapy etc. as explained in the detailed description of the invention.

### BRIEF DESCRIPTION OF THE FIGURES

FIG. 1 shows the prostate tumor growth inhibiting effect of antiprogestin (I) in the LNCaP/IMB human prostate cancer model in mice, compared to a control, the antiandrogen casodex and to castration.

FIG. 2 shows the prostate tumor growth inhibiting effect of antiprogestin (I) in the LNCaP human prostate cancer model in mice, compared to a control, the antiandrogen casodex and to castration as well as to the antiprogestin mifepristone (RU-486).

### DETAILED DESCRIPTION OF THE INVENTION

Antiprogestin (I)—11β-(4-acetylphenyl)-17β-hydroxy-17α-(1,1,2,2,2-pentafluoroethyl)-estra-4,9-dien-3-one—is represented below by formula (I):

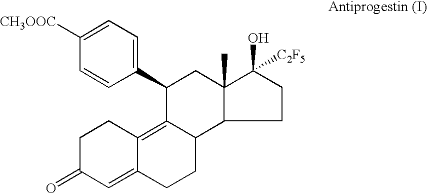

Antiprogestin (I) (or a pharmaceutically acceptable derivative or analogue thereof having comparable activity) is a valuable pharmaceutical agent having strong antiprogestin activity. Antiprogestin (I), as well as further antiprogestins, can be used according to the present invention for the prophylaxis and treatment of prostate cancer and other related prostate diseases like benign prostate hyperplasia.

The term “antiprogestins” in the context of the present invention is intended to primarily comprise all compounds being capable of competitively inhibiting progesterone receptors. However, it should also encompass compounds being capable of inhibiting the biosynthesis of progestins.

It is noted that the antiprogestin or the pharmaceutically acceptable derivative or analogue thereof as defined above can optionally be used according to the present invention in combination with further therapeutic agents.

The derivative or analogue of the preferred antiprogestin according to the present invention as defined above may include, for example, any one of the inventive compounds disclosed in WO 98/34947.

The term “prostate diseases” in the context of the present invention includes, but is not limited to, e.g. prostate cancer and benign prostate hyperplasia.

With respect to the superiority of the present invention over the prior art, it is especially favorable that a targeted treatment of androgen-independent tumors that have relapsed from androgen-ablation therapy is provided.

A further advantage is the exceptionally high bioavailability of antiprogestin (I) or a pharmaceutically acceptable derivative or analogue thereof, allowing it to be administered orally. Oral administration has the advantage of improved convenience and patient compliance.

As a further favorable consequence, antiprogestin (I) or a pharmaceutically acceptable derivative or analogue thereof can be administered in relatively low doses compared to established treatments due to its superior activity as compared to such treatments (see Examples 1 and 2). Lower doses are generally well tolerated and associated with less undesired side effects normally associated with antiprogestins, e.g. side effects resulting from the interaction with glucocorticoid receptors.

Accordingly, a first aspect of the present invention is the use of antiprogestins as defined above, preferably antiprogestin (I) or a pharmaceutically acceptable derivative or analogue thereof, for the manufacture of a medicament for the treatment of prostate diseases, such as prostate cancer and benign prostate hyperplasia.

A second aspect of the present invention relates to a method for the treatment of prostate cancer, benign prostate hyperplasia and other prostate diseases, comprising administering a therapeutically effective amount of at least one antiprogestin, preferably antiprogestin (I) or a pharmaceutically acceptable derivative or analogue thereof to a male mammal, preferably a male human, in need of such treatment.

In another aspect the present invention relates to pharmaceutical compositions comprising at least one antiprogestin as defined above, preferably antiprogestin (I) or a pharmaceutically acceptable derivative or analogue thereof.

Optionally, for the aspects of the present invention, the antiprogestin (I) can be combined with at least one other therapeutic agent, selected from the group consisting of antiandrogens, estrogens, antiestrogens, 5a-reductase inhibitors, cytostatic drugs, antiglucocorticoids, antisense oligonucleotides or similar nucleotides against the antiandrogen receptor, antiangiogenic compounds, gonadotropin releasing hormone agonists and antagonists etc. These active agents may be further combined with additional pharmacologically acceptable and useful agents as defined below, such as commonly used adjuvants, carriers or diluents.

The manufacture of the medicaments may be performed according to methods known in the art. Commonly known and used adjuvants as well as further suitable carriers or diluents may be used. Suitable carriers and adjuvants may be such as recommended for pharmacy, cosmetics and related fields in: Ullmann's Encyclopedia of Technical Chemistry, Vol. 4, (1953), pp. 1-39; Journal of Pharmaceutical Sciences, Vol. 52 (1963), p. 918ff, H. v. Czetsch-Lindenwald, “Hilfsstoffe für Pharmazie und angrenzende Gebiete”; Pharm. Ind. 2, 1961, p. 72ff; Dr. H. P. Fiedler, Lexikon der Hilfsstoffe für Pharmazie, Kosmetik und angrenzende Gebiete, Cantor KG, Aulendorf in Württemberg, 1971.

Antiprogestins suitable for the purposes of the present invention, preferably antiprogestin (I) or a pharmaceutically acceptable derivative or analogue thereof, optionally together with further pharmaceutically active agents as defined above can be incorporated into pharmaceutical compositions according to known methods of preparing galenics for oral, parenteral, e.g. intraperitoneal, intramuscular, subcutaneous or percutaneous application.

They can also be implanted into tissue. Implants can comprise as inert materials e.g. biologically degradable polymers or synthetic silicones such as e.g. silicone rubber.

They can be administered in the form of tablets, pills, dragees, gel capsules, granules, suppositories, implants, injectable sterile aqueous or oily solutions, suspensions or emulsions, ointments, creams or gels.

For the preparation of pharmaceutical compositions for oral administration, the antiprogestins suitable for the purposes of the present invention as defined, preferably antiprogestin (I) or a pharmaceutically acceptable derivative or analogue thereof, optionally in combination with at least one further pharmaceutically active agent as defined above can be admixed with commonly known and used adjuvants and carriers such as for example, gum arabic, talcum, starch, sugars like e.g. mannitose, methyl cellulose, lactose, gelatin, surface-active agents, magnesium stearate, aqueous or non-aqueous excipients, paraffin derivatives, crosslinking agents, dispersants, emulsifiers, lubricants, conserving agents and flavoring agents (e.g., ethereal oils). In the pharmaceutical composition, the antiprogestin may be dispersed in a microparticle, e.g. a nanoparticulate, composition.

In order to further enhance the bioavailability of the active agent, the antiprogestins, preferably antiprogestin (I), suitable for the purposes of the present invention as defined above, optionally in combination with further pharmaceutically active agents as defined above, can also be formulated as cyclodextrin clathrates by reacting them with α-, β- or γ-cyclodextrines or derivatives thereof according to the method as disclosed in PCT/EP 95/02656.

For parenteral administration the antiprogestins suitable for the purposes of the present invention as defined above, preferably antiprogestin (I) or a pharmaceutically acceptable derivative or analogue thereof, optionally in combination with further pharmaceutically active agents as defined above, can be dissolved or suspended in a physiologically acceptable diluent, such as e.g., oils with or without solubilizers, surface-active agents, dispersants or emulsifiers. As oils for example and without limitation, olive oil, peanut oil, cottonseed oil, soybean oil, castor oil and sesame oil may be used.

The amount to be administered (i.e., a “pharmaceutically effective amount”) varies within a broad range and depends on the condition and the patient to be treated as well as the mode of administration. It can cover any amount efficient for the intended treatment. Determining a “pharmaceutically effective amount” is within the purview of the person skilled in the art.

One unit dose represents about 50 to 100 mg, preferably 50 mg active agent(s). For administration to humans, the daily dose of the active agent(s) is about 1 to 500 mg, preferably 50 to 200 mg, most preferably 50 mg, administered once or twice daily.

The pharmaceutical compositions according to the present invention can also be administered via a depot injection or an implant preparation, optionally for sustained delivery of the active agent(s).

The preferred mode of administration is oral administration. The antiprogestins for use according to the invention, and in particular, antiprogestin (I) or a pharmaceutically acceptable derivative or analogue thereof, are particularly suitable for oral administration.

As mentioned above, according to all aspects of the present invention it is also possible to combine at least one antiprogestin as defined above, in particular antiprogestin (I) or a pharmaceutically acceptable derivative or analogue thereof, with at least one further therapeutic agent, such as antiandrogens, estrogens, antiestrogens, 5a-reductase inhibitors, cytostatic drugs, antiglucocorticoids, antisense oligonucleotides or similar nucleotides against the antiandrogen receptor, antiangiogenic compounds, gonadotropin releasing hormone agonists and antagonists etc. Examples for suitable antiandrogens for the combination therapy according to the present invention are casodex, CPA (cyproterone acetate), flutamide etc. Examples for suitable antiestrogens for the combination therapy according to the present invention are SERM's antiestrogens like tamoxifen, raloxifen, 5-(4-{5-[(RS)-(4,4,5,5,5-pentafluoropentyl)sulfinyl]pentyloxy}-phenyl)-6-phenyl-8,9-dihydro-7H-benzocyclohepten-2-ol (WO 00/03979) etc. Examples for suitable estrogens for the combination therapy according to the present invention include estradiol or estradiol derivatives.

Within a combination therapy, the antiandrogen as defined above and the at least one further pharmaceutically active agent may be administered together or separately, at the same time and/or sequentially, under a certain dose regime. Preferably they are administered combined in one unit dose.

For a combination therapy as explained above, the weight ratio of antiprogestin (I) or a pharmaceutically acceptable derivative or analogue thereof to the further therapeutic agent(s) as defined above can vary within a broad range. They can either be present in equal amounts or one component can be present in excess of the other component(s), depending on the nature and activity of the single compounds. Basically, a ratio of 1 to 1000 or 0.0001 to 1 could be used. If the antiprogestin, preferably antiprogestin (I) or a pharmaceutically acceptable derivative or analogue thereof, is combined e.g. with an estrogen, the estrogen may be administered in a much smaller dose than the antiprogestin, i.e., as an example, one unit dose may comprise as little as 0.05 mg of an estrogen and 50 to 100 mg of an antiprogestin, preferably antiprogestin (I) or a pharmaceutically acceptable derivative or analogue thereof.

Experiments conducted show that antiprogestin (I) inhibits prostate cancer cells harboring a mutated androgen receptor in a manner superior to previously known treatments (Example 1). Moreover, it has been shown that antiprogestin (I) inhibits growth of hormone-independent prostate cancer cells in a manner superior to the prior art treatments (Example 2). Further, it has been shown that antiprogestin (I) displays weak anabolic activity at the musculus levator ani. This weak anabolic activity makes the antiprogestin (I) not only suitable for the treatment of already established prostatic deseases but also for the prophylaxis of such deseases without the unfavorable side-effects of commonly available endocrine treatment options (e.g. antiandrogens or GnRH super agonists) for prostate deseases. The models in these tests represent standard in vivo animal models for prostate cancer. The following examples are not to be understood as a limitation.

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever.

In the foregoing and in the following examples, all temperatures are set forth uncorrected in degrees Celsius and, all parts and percentages are by weight, unless otherwise indicated.

### EXAMPLE 1

### LNCaP/IMB Androgen Independent Human Prostate Xenograft

Materials and Methods

LNCaP/MIB prostate cancer cells (i.e., LNCAP prostate cancer cells, obtainable from ATCC and modified according to Culig et al., Br. J. Cancer 1999 September; 81(2), pp. 242 -251, resulting in a hormone-independent subline) obtained from cell culture and suspended in matrigel, were implanted s.c. in the inguinal region of male nude mice (M&B). Treatment was started when the tumors were approximately 25 mm² in size. Treatment was continued until progression of the tumors. Experimental groups were: 1) control (vehicle), 2) castration, 3) the antiandrogen casodex, 30 mg/kg s.c. and 4) 11β-(4-acetylphenyl)-17β-hydroxy-17α-(1,1, 2,2,2-pentafluoroethyl)-estra-4,9-dien-3-one (antiprogestin (I)), 50 mg/kg s.c., 5 times weekly. The tumor area was determined by caliper measurements. The Kruskal Wallis test was used for statistical analysis of intergroup differences of mean values. The tumor weight was determined at the end of the experiment.

Results

Compared to the rapid growth of the control and also compared to castration and the treatment with the antiandrogen casodex, the antiprogestin (I) according to the present invention caused a pronounced and lasting tumor growth inhibition over a prolonged period of time. Thus, the treatment with antiprogestin (I) exerted an antitumor effect superior to that of either castration or the antiandrogen casodex.

Conclusion

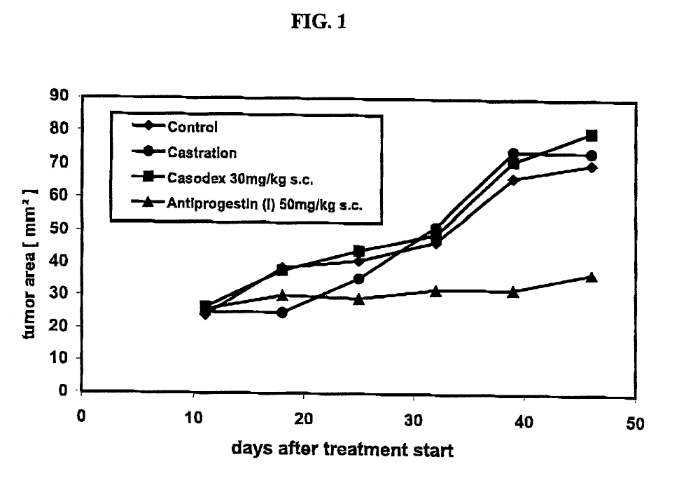

The antiprogestin (I) proved to be a potent inhibitior of the growth of LNCaP/IMB prostate tumor. The effect on tumor growth was superior to that of the antiandrogen casodex. FIG. 1 clearly shows that the antiprogestin (I) used according to the present invention exerts a strong inhibiting effect on tumor development in the LNCaP/IMB (a hormone independent LNCAP subline) human prostate cancer model. Surprisingly, antiprogestin (I) is significantly superior in its activity to the antiandrogen casodex and even to castration which has always been considered as the only effective treatment for prostate cancer.

### EXAMPLE 2

### LNCaP Human Prostate Cancer Model

Materials and Methods

Male nude mice (M&B) were supplemented with testosterone pellets (Innovative Research of America). LNCaP prostate cancer cells (obtainable from ATCC), obtained from cell culture and suspended in matrigel, were implanted s.c. in the inguinal region of the mice. Treatment was started when the tumors were approximately 25 mm² in size. Treatment was continued until progression of the tumors, Experimental groups were: 1) control (vehicle), 2) castration, 3) the antiandrogen casodex, 30 mg/kg s.c., 4) the antiprogestin mifepristone (RU-486), 10 mg/kg sec., and 5) 11β-(4-acetylphenyl)-17β-hydroxy-17α-(1,1,2,2,2-pentafluoroethyl)-estra-4,9-dien-3-one (antiprogestin (I)), 10 mg/kg sc., 5 times weekly. The tumor area was determined by caliper measurements. The Kruskal Wallis test was used for statistical analysis of intergroup differences of mean values. The tumor weight was determined at the end of the experiment.

Results

Compared to the rapid growth of the control, castration caused a pronounced tumor growth inhibition. The treatment with antiprogestin (I) exerted an antitumor effect significantly superior to that of either the antiandrogen casodex or the antiprogestin mifepristone.

Conclusion

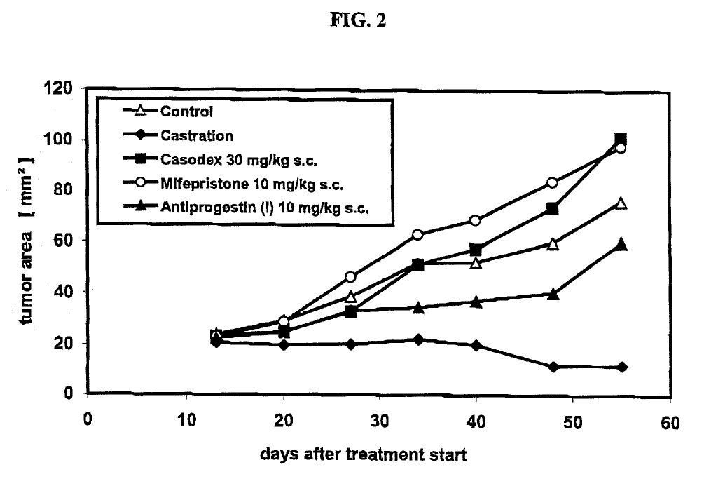

The antiprogestin (I) proved to be an inhibitior of the growth of LNCaP prostate tumor cells. The effect on tumor growth was superior that of the antiandrogen casodex and the other antiprogestin, mifepristone, and initially equally effective as castration. FIG. 2 shows the effective tumor inhibition by antiprogestin (I) in the LNCaP (mutated androgen receptor) human prostate cancer model, compared to the antiandrogen casodex and the other antiprogestin, mifepristone (RU486), as well as castration. Mice treated with antiprogestin (I) showed an initially delayed tumor growth, whereas casodex and mifepristone were totally ineffective. Thus, antiprogestin (I) used according to the present invention is superior to known pharmaceutical treatments of prostate cancer.

The entire disclosure of all applications, patents and publications, cited herein and of corresponding U.S. Provisional Application No. 60/293,194, filed May 25, 2001 is incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Use of the antiprogestin 11β-(4-acetylphenyl)-17β-hydroxy-17α-(1,1,2,2,2-pentafluoroethyl)-estra-4,9-dien-3-one or a pharmaceutically acceptable derivative or analogue thereof for the manufacture of a medicament for the treatment of a prostate disease in a male mammal.

2. Use of the antiprogestin 11β-(4-acetylphenyl)-17β-hydroxy-17α-(1,1,2,2,2-pentafluoroethyl)-estra-4,9-dien-3-one or a pharmaceutically acceptable derivative or analogue thereof for the manufacture of a medicament for the prophylaxis and treatment of a prostate disease in a male mammal.

3. Use of the antiprogestin 11β-(4-acetylphenyl)-17β-hydroxy-17α-(1,1,2,2,2-pentafluoroethyl)-estra-4,9-dien-3-one or a pharmaceutically acceptable derivative or analogue thereof for the manufacture of a medicament for the prophylaxis of a prostate disease in a male mammal.

4. Use according to any preceeding claim, wherein the prostate disease is prostate cancer or benign prostate hyperplasia.

5. Use according to any preceeding claim, wherein the male mammal is a male human.

6. Use according to any preceding claim, wherein the medicament is administered orally.

7. Use according to any preceding claim, wherein the antiprogestin 11β-(4-acetylphenyl)-17β-hydroxy-17α-(1,1,2,2,2-pentafluoroethyl)-estra-4,9-dien-3-one or a pharmaceutically acceptable derivative or analogue thereof is administered in a unit dose of 50 to 100 mg.

8. Use according to any preceding claim wherein the antiprogestin 11β-(4-acetylphenyl)-17β-hydroxy-17α-(1,1,2,2)-2-pentafluoroethyl)-estra-4,9-dien-3-one or a pharmaceutically acceptable derivative or analogue thereof is combined with at least one further therapeutic agent.

9. Use according to claim 8, wherein the further therapeutic agent is selected from the group consisting of antiandrogens, estrogens, antiestrogens, 5a-reductase inhibitors, cytostatic drugs, antiglucocorticoids, antisense oligonucleotides or similar nucleotides against the antiandrogen receptor, antiangiogenic compounds, gonadotropin releasing hormone agonists and antagonists.

10. Pharmaceutical composition comprising the antiprogestin 11β-(4-acetylphenyl)-17β-hydroxy-17α-(1,1,2,2,2-pentafluoroethyl)-estra-4,9-dien-3-one or a pharmaceutically acceptable derivative or analogue thereof in an amount sufficient for the treatment of a prostate disease.

11. Pharmaceutical composition comprising the antiprogestin 11β-(4-acetylphenyl)-17β-hydroxy-17α-(1,1,2,2,2-pentafluoroethyl)-estra-4,9dien-3-one or a pharmaceutically acceptable derivative or analogue thereof in an amount sufficient for the prophylaxis and treatment of a prostate disease.

12. Pharmaceutical composition comprising the antiprogestin 11β-(4-acetylphenyl)-17β-hydroxy-17α-(1,1,2,2,2-pentafluoroethyl)-estra-4,9-dien-3-one or a pharmaceutically acceptable derivative or analogue thereof in an amount sufficient for the prophylaxis of a prostate disease.

13. Pharmaceutical composition according to any one of claims 10 to 12, wherein the antiprogestin 11β-(4-acetylphenyl)-17β-hydroxy-17α-(1,1,2,2,2-pentafluoroethyl)estra-4,9-dien-3-one or a pharmaceutically acceptable derivative or analogue thereof is present in a unit dose of 50 to 100 mg.

14. Pharmaceutical composition according to any one of claims 10 to 13, wherein the prostate disease is prostate cancer or benign prostate hyperplasia.

15. Pharmaceutical composition according to any one of claims 10 to 14, wherein the antiprogestin 11β-(4-acetylphenyl)-17β-hydroxy-17α-(1,1,2,2,2-pentafluoro-ethyl)-estra-4,9-dien-3-one or a pharmaceutically acceptable derivative or analogue thereof is combined with at least one further therapeutic agent.

16. Pharmaceutical composition according to claim 15, wherein the further therapeutic agent is selected from the group consisting of antiandrogens, estrogens, antiestrogens, 5a-reductase inhibitors, cytostatic drugs, antiglucocorticoids, antisense oligonucleotides or similar nucleotides against the antiandrogen receptor, antiangiogenic compounds, gonadotropin releasing hormone agonists and antagonists.
